(19) United States
(12) Patent Application Publication
(10) Pub. No.: US 2026/0345711 A1
(43) Pub. Date: Nov. 5, 2026

(54) Methods for Binding to and Inhibiting the Activity of the SC2 RNA-Dependent RNA Polymerase (6M71) using Tulsi Extracts

(71) Applicant: Dawn Solomon, Missouri, TX (US)

(72) Inventor: Dawn Solomon, Missouri, TX (US)

(21) Appl. No.: 19/196,139

(22) Filed: May 1, 2025

(51) Int. Cl. A61K 36/53; A61K 36/48

(52) U.S. Cl. CPC A61K 36/53; A61K 36/48; A61K 2236/331

(57) ABSTRACT

Methods for binding to and inhibiting the activity of the SC2 RNA-dependent RNA polymerase (6M71) using tulsi extracts from Ocimum x africanum are disclosed. One method includes extracting tulsi extracts and administering them to a person through various delivery mechanisms, such as tea, intravenous (IV) fluid, or encapsulated capsules. Another method includes encapsulating fenugreek seeds and neem with tulsi extracts in capsules to enhance the binding and inhibition of SC2 RNA-dependent RNA polymerase (6M71).

### BACKGROUND

This disclosure relates to a system and method for reducing oxidative stress in the blood. The system and method is also for binding to and inhibiting the activity of SARS-CoV-2 proteins, binding to and inhibiting the activating of the SC2 RNA-dependent RNA polymerase (6M71), binding to and inhibiting the activity of the SC2 main protease (6Y84), dissolving graphene in the blood, and breaking down clotting structures in the blood. For purposes of this disclosure, specific examples of the systems and methods are discussed. However, such discussion is solely exemplary and not limiting.

Oxidative stress is a condition characterized by an imbalance between the production of free radicals and the body's ability to detoxify their harmful effects through antioxidants. This imbalance leads to damage of cellular components, contributing to the aging process and the development of various diseases, including cardiovascular diseases, diabetes, neurodegenerative disorders, and cancer. Given its significant health implications, there is a growing interest in natural and effective methods to mitigate oxidative stress in the blood.

According to a paper published by the National Institute of Health entitled, “Oxidative Stress and Hyper-Inflammation as Major Drivers of Severe COVID-19 and Long COVID: Implications for the Benefit of High-Dose Intravenous Vitamin C,” oxidative stress and inflammation are key drivers of severe COVID-19 and Long COVID. They contribute to hyperinflammation, coagulopathy, and immune dysfunction. Additionally, vitamin C deficiency is common in COVID-19 patients, leading to severe pathological consequences. Vitamin C is a potent antioxidant and co-factor for many enzymatic processes. Further, intravenous Vitamin C: High-dose IV vitamin C has shown benefits in 12 studies with 1578 COVID- 19 patients, improving oxygenation, reducing inflammation, and speeding recovery. The paper goes on to state that in long COVID, persistent inflammation, thrombosis, and dysregulated immune response are major contributors. Oxidative stress and inflammation disrupt tissue, blood flow, and neurotransmitter metabolism. The authors of the paper found IV vitamin C may reduce the risk of severe COVID-19 and Long COVID, alleviate symptoms like fatigue and cognitive dysfunction, and improve overall recovery, but stated that more research was needed.

However, while high-dose intravenous Vitamin C has shown promising benefits in improving oxygenation, reducing inflammation, and speeding recovery in COVID-19 patients, it is important to consider potential downsides. One concern is the risk of kidney stones, as excessive Vitamin C can increase oxalate levels in the urine, potentially leading to stone formation. Additionally, high doses of Vitamin C may cause gastrointestinal disturbances such as diarrhea and abdominal cramps. There is also the possibility of interactions with other medications, which could alter their effectiveness or cause adverse effects. Furthermore, the long-term safety of high-dose Vitamin C treatment remains uncertain, and, as the author of the NHS piece stated, more research is needed to fully understand its implications.

Some tulsis have been shown to reduce oxidative stress. However, the bioavailability of the active compounds in Tulsi can be limited. This means that the body may not absorb and utilize these compounds efficiently.

As such, it would be advantageous to have a new system and method for reducing oxidative stress in the blood.

### SUMMARY

The disclosure teaches a system and method for reducing oxidative stress in the blood. The system can comprise a mixture of tulsi extracts and fenugreek seed extracts, which can be contained in various forms such as ground leaves and seeds, dried and broken leaves, or suspended in intravenous (IV) fluid. The mixture may also include neem extracts, which can be contained in neem leaves and mixed with the tulsi and fenugreek extracts. The system can be encapsulated in a capsule or tea bag for easy administration.

The method teaches combining tulsi extracts with fenugreek seed extracts to form a mixture that is administered to a person to reduce oxidative stress. The mixture may also include neem extracts to enhance the effectiveness of the treatment. The tulsi extracts can be derived from different species such as Ocimum x africanum or Ocimum sanctum.

The disclosure also teaches a system and methods for binding to and inhibiting the activity of the SARS-CoV-2 surface spike protein (6VSB) using tulsi extracts from Ocimum x africanum. In one instance, the method involves extracting tulsi extracts and administering them to a person through various delivery mechanisms, such as tea, intravenous (IV) fluid, or encapsulated capsules. In one embodiment, the method can include boiling Ocimum x africanum to extract tulsi extracts and using the water from boiling as tea, which the person drinks to administer the tulsi extracts. In another embodiment, the method can involve using IV fluid as a delivery mechanism for tulsi extracts.

In another instance, the method involves encapsulating Ocimum x africanum in a capsule for administering to a person. The capsule may also contain fenugreek seeds and neem to enhance the binding and inhibition of SARS-CoV-2 surface spike protein (6VSB). The Ocimum x africanum can be in powder form or derived from leaves.

A system for binding to and inhibiting the activity of the SARS-CoV-2 surface spike protein (6VSB) is also disclosed, namely an IV fluid. The IV fluid can comprise 0.9% saline and tulsi extracts from Ocimum x africanum. The IV fluid may also include fenugreek seed extracts and/or neem extracts to enhance the binding and inhibition of SARS-CoV-2 surface spike protein (6VSB).

The disclosure teaches methods for binding to and inhibiting the activity of the SC2 RNA-dependent RNA polymerase (6M71) using tulsi extracts from Ocimum x africanum. In one instance, the method involves extracting tulsi extracts and administering them to a person through various delivery mechanisms, such as tea, intravenous (IV) fluid, or encapsulated capsules. In one embodiment, the method can include boiling Ocimum x africanum to extract tulsi extracts and using the water from boiling as tea, which the person drinks to administer the tulsi extracts. In another embodiment, the method can involve using IV fluid as a delivery mechanism for tulsi extracts.

In another instance, the method involves encapsulating Ocimum x africanum in a capsule for administering to a person. The capsule may also contain fenugreek seeds and neem to enhance the binding and inhibition of SC2 RNA-dependent RNA polymerase (6M71). The Ocimum x africanum can be in powder form or derived from leaves.

The disclosure also teaches methods for binding to and inhibiting the activity of the SC2 main protease (6Y84) using tulsi extracts from Ocimum x africanum. In one instance, the method involves extracting tulsi extracts and administering them to a person through various delivery mechanisms, such as tea, intravenous (IV) fluid, or encapsulated capsules. In one embodiment, the method can include boiling Ocimum x africanum to extract tulsi extracts and using the water from boiling as tea, which the person drinks to administer the tulsi extracts. In another embodiment, the method can involve using IV fluid as a delivery mechanism for tulsi extracts.

In another instance, the method involves encapsulating Ocimum x africanum in a capsule for administering to a person. The capsule may also contain fenugreek seeds and neem to enhance the binding and inhibition of SC2 main protease (6Y84). The Ocimum x africanum can be in powder form or derived from leaves.

The disclosure teaches methods for dissolving graphene in blood using tulsi extracts from Ocimum x africanum. In one instance, the method involves extracting tulsi extracts and administering them to a person through various delivery mechanisms, such as tea, intravenous (IV) fluid, or encapsulated capsules. In one embodiment, the method can include boiling Ocimum x africanum to extract tulsi extracts and using the water from boiling as tea, which the person drinks to administer the tulsi extracts. In another embodiment, the method can involve using IV fluid as a delivery mechanism for tulsi extracts.

In another instance, the method involves encapsulating Ocimum x africanum in a capsule for administering to a person. The capsule may also contain fenugreek seeds and neem to enhance the dissolving of graphene in blood. The Ocimum x africanum can be in powder form or derived from leaves.

The disclosure teaches methods for dissolving graphene in blood using tulsi extracts from Ocimum x africanum. In one instance, the method involves extracting tulsi extracts and administering them to a person through various delivery mechanisms, such as tea, intravenous (IV) fluid, or encapsulated capsules. In one embodiment, the method can include boiling Ocimum x africanum to extract tulsi extracts and using the water from boiling as tea, which the person drinks to administer the tulsi extracts. In another embodiment, the method can involve using IV fluid as a delivery mechanism for tulsi extracts. In another instance, the method involves encapsulating Ocimum x africanum in a capsule for administering to a person. The capsule may also contain fenugreek seeds and neem to enhance the dissolving of graphene in blood. The Ocimum x africanum can be in powder form or derived from leaves.

### BRIEF DESCRIPTION OF THE DRAWINGS

FIG. 1 illustrates tulsi.

FIG. 2 illustrates fenugreek seeds.

FIG. 3 illustrates neem.

FIG. 4 illustrates a delivery mechanism comprising a capsule.

FIG. 5 illustrates a delivery mechanism comprising a tea.

FIG. 6 illustrates a delivery mechanism comprising intravenous (IV) fluid.

FIG. 7 illustrates a method for reducing oxidative stress in the blood.

FIG. 8 illustrates a method for binding to and inhibiting the activity of SARS-CoV-2 Surface Spike Protein (6VSB).

FIG. 9 illustrates a second method for binding to and inhibiting the activity of SARS-CoV-2 Surface Spike Protein (6VSB).

FIG. 10 illustrates a method for binding to and inhibiting the activity of SC2 RNA-dependent RNA Polymerase (6M71).

FIG. 11 illustrates a second method for binding to and inhibiting the activity of SC2 RNA-dependent RNA Polymerase (6M71).

FIG. 12 illustrates a method for binding to and inhibiting the activity of SC2 Main Protease (6Y84).

FIG. 13 illustrates a second method for binding to and inhibiting the activity of SC2 Main Protease (6Y84).

FIG. 14 illustrates a method for dissolving graphene in blood.

FIG. 15 illustrates a second method for dissolving graphene in blood.

FIG. 16 illustrates a third method for dissolving graphene in blood.

FIG. 17 illustrates a method for breaking down clotting structures in blood.

FIG. 18 illustrates a second method for breaking down clotting structures in blood.

FIG. 19 illustrates a third method for breaking down clotting structures in blood.

### DETAILED DESCRIPTION OF THE DRAWINGS

This disclosure teaches systems and methods utilizing tulsi, both alone and in combination with other herbs, to achieve various health benefits. For example, the disclosure teaches a system and method for reducing oxidative stress in the blood. The disclosure also teaches methods for for binding to and inhibiting the activity of SARS-CoV-2 proteins, binding to and inhibiting the activating of the SC2 RNA-dependent RNA polymerase (6M71), binding to and inhibiting the activity of the SC2 main protease (6Y84). Further, the application teaches methods for dissolving graphene in the blood, and also methods for breaking down clotting structures in the blood. For purposes of this disclosure, specific examples of the systems and methods are discussed. The disclosure presents descriptions to enable any person skilled in the art to make and use the inventions as claimed and provides descriptions in the context of the particular examples discussed below. Variations will be readily apparent to those skilled in the art. As such, in the interest of clarity, the disclosure does not discuss all possible variations, nor does it describe not all features of an actual implementation, which would be known and understood to be there by one of ordinary skill in the art. It will be appreciated that in the development of any such actual implementation (as in any development project), design decisions must be made to achieve the designers'specific goals (e.g., compliance with system- and business-related constraints), and that these goals will vary from one implementation to another. It will also be appreciated that such development effort might be complex and time-consuming but would nevertheless be a routine undertaking for those of ordinary skill in the field of the art having the benefit of this disclosure. Accordingly, the claims appended hereto are not intended to be limited by the disclosed embodiments but are to be accorded their widest scope consistent with the principles and features disclosed herein. Additionally, when specific ratios are given, unless otherwise specified, a tolerance of ten percent is implicit within all ratios taught, such that if a specific ratio is provided, a ten percent tolerance is implied. If a ratio is claimed, such ten percent tolerance is a part of such claim.

Tulsi, also known as holy basil, is a revered herb in traditional medicine, known for its potent antioxidant properties. This application specifically discusses the use of different types of tulsi, including Ocimum x africanum and Ocimum sanctum, which are highlighted for their effectiveness in reducing oxidative stress, inhibiting the activity of various SARS-CoV-2 proteins, dissolving graphene in blood, and breaking down clotting structures in blood.

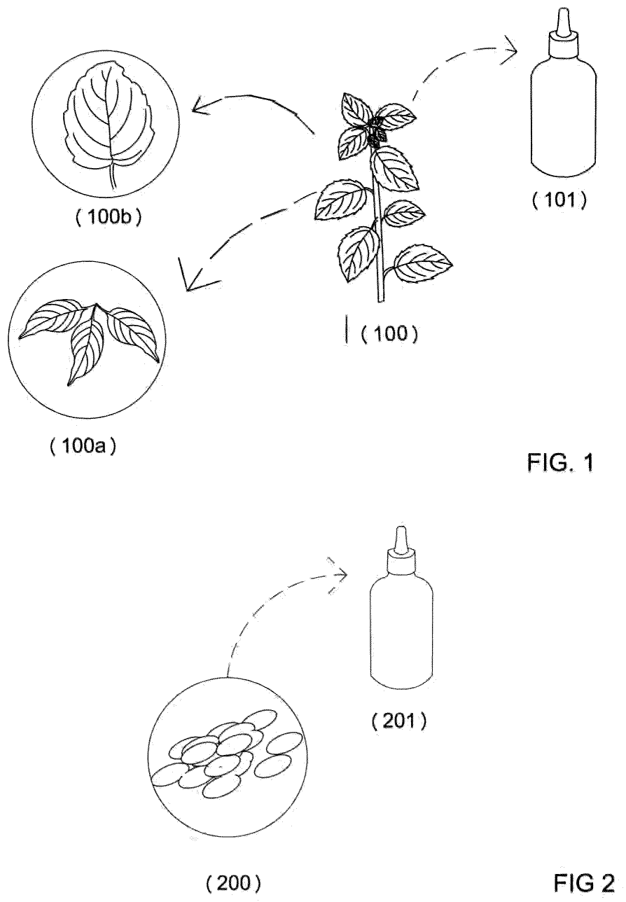

FIG. 1 illustrates tulsi 100. Tulsi 100, some species of which are known as basil or holy basil, comprises tulsi extracts 101. Tulsi extracts 101 include but are not limited to aqueous extract, ethanol in extract, methanolic extract, ethyl acetate extract, tulsi leaf extract, and essential oils. Ocimum x africanum 100a, a hybrid variety between basil (Ocimum basilicum) and American basil (Ocimum americanum), is unique in that it has the highest concentrations of essential oils and methanolic extract. Another variety that can be used is Ocimum sanctum 100b

FIG. 2 illustrates fenugreek seeds 200. Fenugreek seeds 200 comprise fenugreek seed extracts 201. Tulsi extracts include but are not limited to methanolic extract, ethanolic extract, dichloromethane extract, acetone extract, hexane extract, ethyl acetate extract, and galactomannan fiber extract.

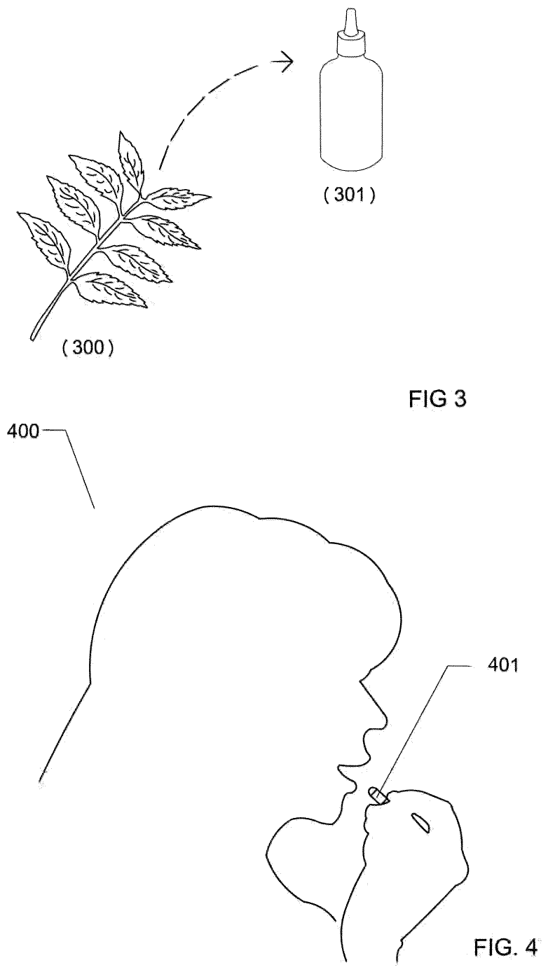

FIG. 3 illustrates neem leaves 300. Neem leaves 300 comprise neem leaf extract 301. Neem leaf extracts 301 contain compounds such as nimbin, quercetin, and azardirachtin.

FIG. 4 illustrates a delivery mechanism 400 comprising a capsule 401. Capsule 401 can be used to deliver various embodiments using tulsi extracts 101 as described in this disclosure. In one embodiment, tulsi 100 can be encapsulated alone in capsule 401. In a preferred embodiment, Ocimum x africanum 100a is the tulsi encapsulated alone. Such capsule form allows for easy administration and ensures that the active compounds are efficiently absorbed by a body when digested.

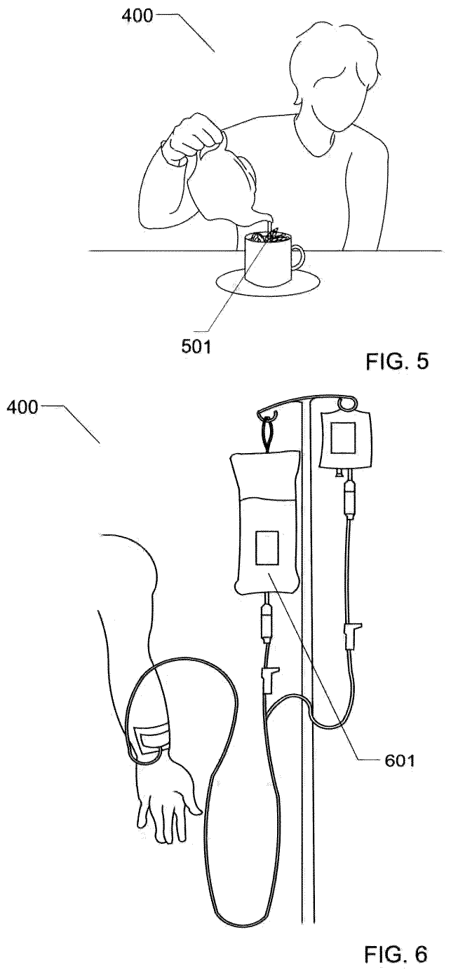

FIG. 5 illustrates a delivery mechanism 400 comprising a tea 501. In one embodiment, tulsi extracts from Ocimum x africanum 100a are brewed alone to create a tea. Brewing in tea 501 allows for easy administration and ensures that the active compounds are efficiently absorbed by the body when the tea is consumed by a person.

Another embodiment involves a mixture 500 of tulsi extracts 101 with fenugreek seed extracts 201 brewed together to make a tea 501. The preferred ratio for this mixture is 2:1, as disclosed in the claims. This combination enhances the antioxidant properties of the treatment, providing a synergistic effect that significantly reduces oxidative stress in the blood.

In a further embodiment, neem leaves 300 can be added to the mixture 500 of tulsi 100 and fenugreek seed extracts 201. The preferred ratio for this mixture is 4:2:1, ensuring maximum effectiveness. The inclusion of neem leaves 300 provides additional health benefits and contributes to the overall efficacy of the method.

The mixtures described above can be made into a tea in various forms, including as whole leaves and seeds, ground leaves and seeds, dried and broken leaves and seeds, or various combinations there of. The mixture can be brewed loosely or encapsulated in a tea bag.

FIG. 6 illustrates a delivery mechanism 400 comprising intravenous (IV) fluid 601. The intravenous fluid 601 can comprise 0.9% saline; and various extracts as described below.

This IV fluid system is to administer the various extracts into the bloodstream. The extracts can be obtained from various species of tulsi 100 such as Ocimum x africanum 100a or Ocimum sanctum 100b, from fenugreek seeds 200, and/or from neem leaves 300. The extraction process can in involve boiling various leaves and/or seeds to obtain the extracts, which can then be suspended in the IV fluid 601.

In one embodiment, the IV fluid 601 contains tulsi extracts 101 from Ocimum x africanum 100a. Another embodiment involves a mixture of tulsi extracts 101 and fenugreek seed extracts 201 in the IV fluid. The preferred ratio for this mixture of tulsi extracts 101 to fenugreek seed extracts 201 is 2:1.

In a further embodiment, neem leaf extracts 301 can be added to the mixture 500 of tulsi extracts 101 and fenugreek seed extracts 201. The preferred ratio for this mixture of tulsi extracts 101, fenugreek seed extracts 201, and neem leaf extracts 301 is 4:2:1, ensuring maximum effectiveness. The inclusion of neem leaf extracts 301 provides additional health benefits and contributes to the overall efficacy of the method.

By combining these extracts in the IV fluid 601, the system offers a comprehensive approach to reducing oxidative stress, inhibiting viral activity, and improving blood health. This innovative delivery mechanism ensures that the active compounds are efficiently absorbed into the bloodstream, providing a natural and effective solution to combat various health issues.

Another embodiment involves a mixture of tulsi 100 with fenugreek seed extracts 201. The preferred ratio for this mixture is 2:1, as disclosed in the claims. This combination enhances the antioxidant properties of the treatment, providing a synergistic effect that significantly reduces oxidative stress in the blood.

In a further embodiment, neem leaf extracts 301 are added to the mixture 500 of tulsi 100 and fenugreek seed extracts 201. The preferred ratio for this mixture is 4:2:1, ensuring maximum effectiveness. The inclusion of neem leaf extracts 301 provides additional health benefits and contributes to the overall efficacy of the method.

The mixture 500 can be administered in various forms, including ground leaves and seeds, dried and broken leaves, or suspended in intravenous (IV) fluid. For ease of administration, the mixture 500 can be encapsulated in capsule 401 or tea bag. The IV fluid 601 option provides a direct and efficient delivery mechanism, ensuring that the active compounds are rapidly absorbed into the bloodstream.

Other delivery mechanisms are contemplated included including absorbable implants, absorbable patches, and suppositories. In such delivery mechanisms, possible embodiments are similar to other delivery mechanisms, i.e., tulsi extracts 101 alone, tulsi extracts 101 with fenugreek seed extracts 201, or tulsi extracts 101 with fenugreek seed extracts 201 and neem leaf extracts 301. Preferred ratios are the same as disclosed above in other delivery mechanisms.

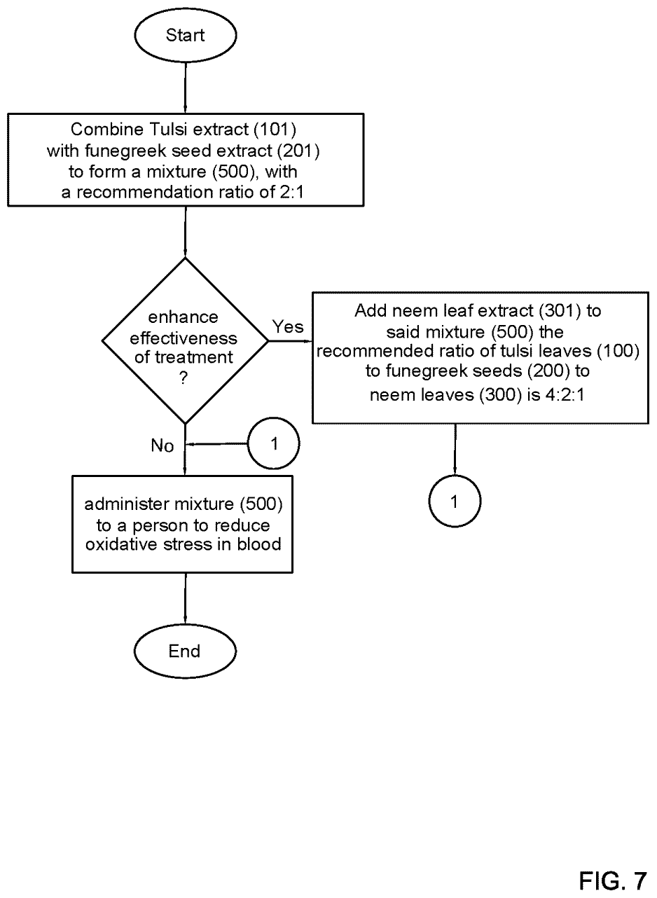

FIG. 7 illustrates a method for reducing oxidative stress in the blood. This method involves combining tulsi extracts 101 with fenugreek seed extracts 201 to produce unexpected significant antioxidant effects to mitigate the harmful effects of oxidative stress. Oxidative stress is a condition characterized by an imbalance between the production of free radicals and the body's ability to detoxify their harmful effects through antioxidants. This imbalance leads to damage of cellular components, contributing to the aging process and the development of various diseases, including cardiovascular diseases, diabetes, neurodegenerative disorders, and cancer.

The method can with the extraction of tulsi extracts 101 from species such as Ocimum x africanum 100a or Ocimum sanctum 100b. These extracts can be combined with fenugreek seed extracts 201 to form a mixture 500. One embodiment of the method involves using only tulsi extracts 101 and fenugreek seed extracts 201, with a recommended ratio of 2:1. This mixture can be administered to a person to reduce oxidative stress in the blood.

In another embodiment, neem leaf extracts 301 can be added to the mixture 500 to enhance the effectiveness of the treatment. The ratio of tulsi leaves 100 to fenugreek seeds 200 to neem leaves 300 is carefully calibrated to ensure maximum effectiveness, with a recommended ratio of 4:2:1. The inclusion of neem leaf extracts 301 provides additional health benefits and contributes to the overall efficacy of the method.

The mixture can be administered in various forms, including ground leaves and seeds, dried and broken leaves, or suspended in intravenous (IV) fluid 601. For ease of administration, the mixture 500 can be encapsulated in capsule 401 or tea bag. The IV fluid 500 option provides a direct and efficient delivery mechanism, ensuring that the active compounds are rapidly absorbed into the bloodstream.

By combining these extracts, the method effectively reduces oxidative stress in the blood, thereby mitigating the damage caused by free radicals and improving overall health outcomes. This innovative approach offers a natural and effective solution to combat oxidative stress.

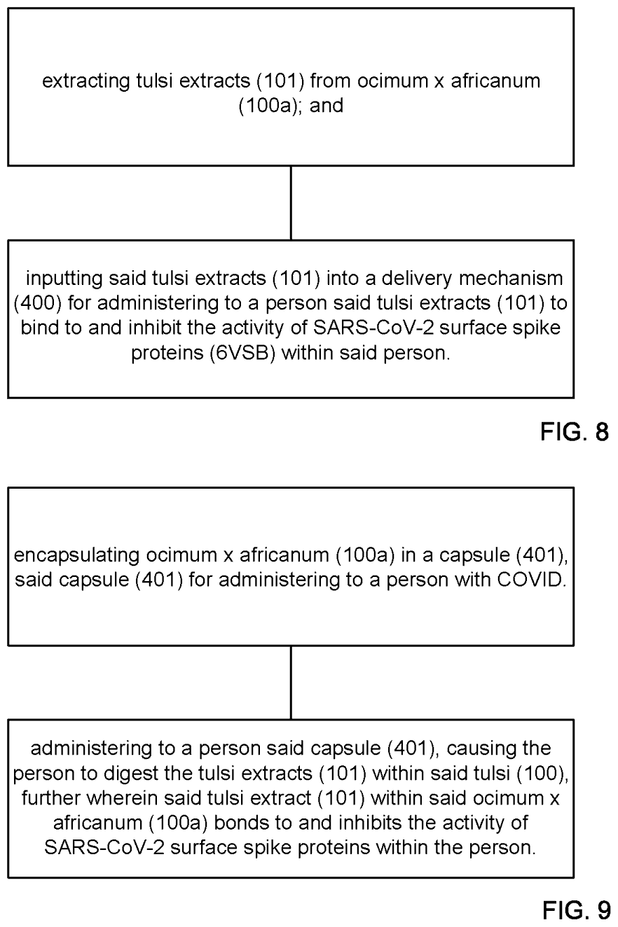

FIG. 8 illustrates a method for binding to and inhibiting the activity of SARS-CoV-2 Surface Spike Protein (6VSB). This method involves extracting tulsi extracts 101 from tulsi leaves 100 such as Ocimum x africanum 100a and inputting these extracts into a delivery mechanism for administering to a person. The tulsi extracts 101 can then bind to and inhibit the activity of SARS-CoV-2 surface spike proteins (6VSB) within the person. In one embodiment, the extraction process can include boiling the tulsi 100, and the delivery mechanism 400 can be the water used in boiling, forming a tea 501. Administering the tulsi extracts 101 involves the person drinking the tea. Alternatively, the delivery mechanism 400 can be intravenous (IV) fluid 601.

FIG. 9 illustrates a second method for binding and inhibiting the activity of SARS-COV-2 Surface Spike Protein (6VSB). Another method for binding to and inhibiting the activity of SARS-CoV-2 surface spike protein (6VSB) involves encapsulating tulsi 100 such as Ocimum x africanum 100a in capsule 401. This capsule 401 can be administered to a person, allowing the tulsi extracts 101 within Ocimum x africanum 100a to bind to and inhibit the activity of SARS-CoV-2 surface spike proteins within the person. Capsule 401 may also contain fenugreek seeds 200 and neem 300 to enhance the binding and inhibition process. The contents of capsule 401 can be in powder form or be broken or shredded leaves and/or seeds respectively.

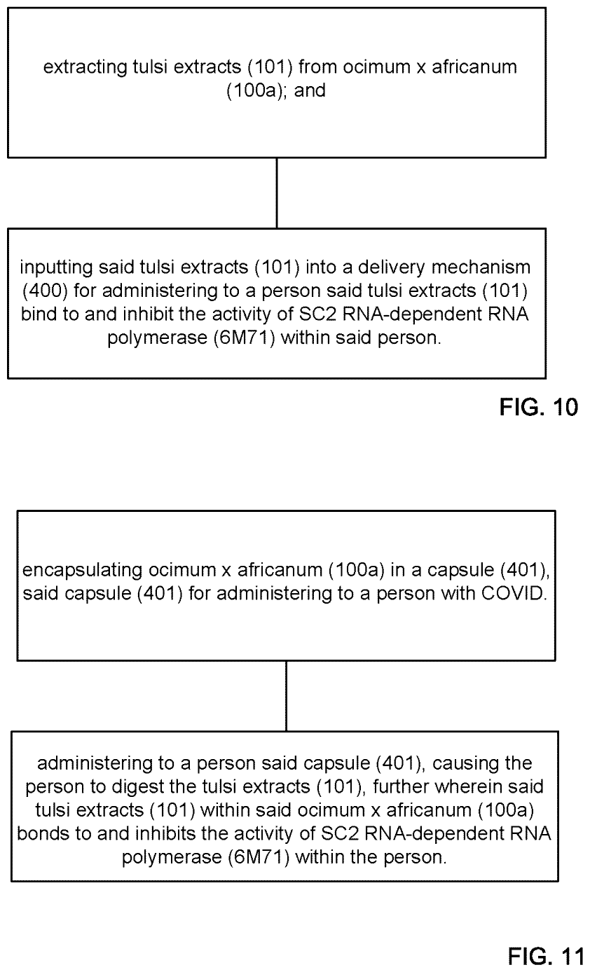

FIG. 10 illustrates a method for binding to and inhibiting the activity of SC2 RNA-dependent RNA Polymerase (6M71). This method involves extracting tulsi extracts 101 from tulsi 100 such as Ocimum x africanum 100a and inputting these extracts into a delivery mechanism 400 for administering to a person. The tulsi extracts 101 can then bind to and inhibit the activity of SC2 RNA-dependent RNA Polymerase (6M71) within the person. In one embodiment, the extraction process can include boiling the tulsi 100, and the delivery mechanism 400 can be the water used in boiling, forming a tea 501. Administering the tulsi extracts 101 involves the person drinking the tea 501. Alternatively, the delivery mechanism 400 can be intravenous (IV) fluid 601.

FIG. 11 illustrates a second method for binding and inhibiting the activity of SC2 RNA-dependent RNA Polymerase (6M71). Another method for binding to and inhibiting the activity of SC2 RNA-dependent RNA Polymerase (6M71) involves encapsulating tulsi 100 such as Ocimum x africanum 100a in capsule 401. This capsule 401 can be administered to a person, allowing the tulsi extracts 101 within Ocimum x africanum 100a to bind to and inhibit the activity of SC2 RNA-dependent RNA Polymerase (6M71) within the person. Capsule 401 may also contain fenugreek seeds 200 and neem 300 to enhance the binding and inhibition process. The contents of Capsule 401 can be in powder form or be broken or shredded leaves and/or seeds respectively.

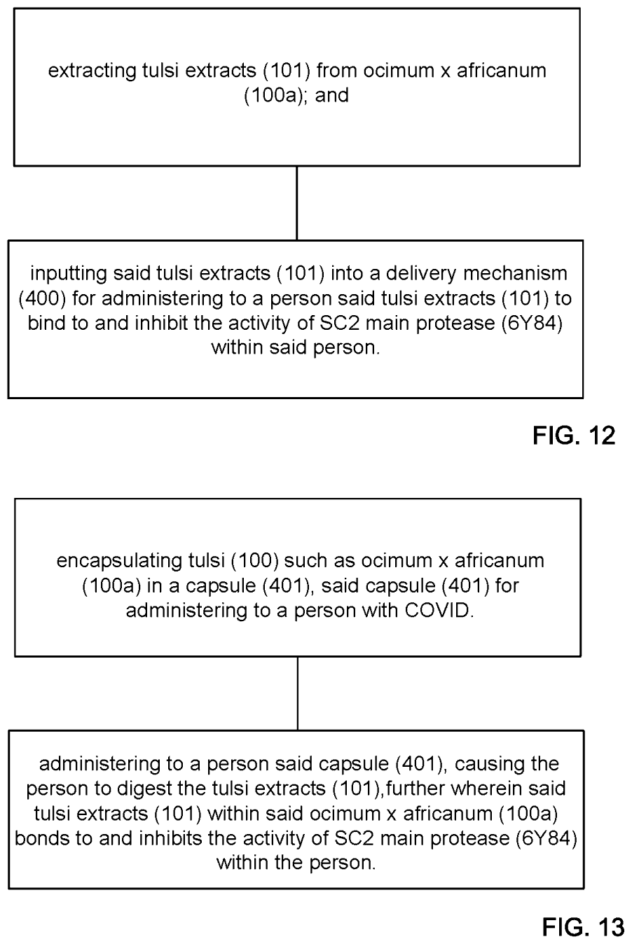

FIG. 12 illustrates a method for binding to and inhibiting the activity of SC2 Main Protease (6Y84). This method involves extracting tulsi extracts 101 from tulsi 100 such as Ocimum x africanum 100a and inputting these extracts into a delivery mechanism 400 for administering to a person. The tulsi extracts 101 can then bind to and inhibit the activity of SC2 Main Protease (6Y84) within the person. In one embodiment, the extraction process can include boiling the tulsi 100, and the delivery mechanism 400 can be the water used in boiling, forming a tea 501. Administering the tulsi extracts 101 involves the person drinking the tea 501. Alternatively, the delivery mechanism can be intravenous (IV) fluid 601.

FIG. 13 illustrates a second method for binding and inhibiting the activity of SC2 Main Protease (6Y84). Another method for binding to and inhibiting the activity of SC2 Main Protease (6Y84) involves encapsulating tulsi 100 such as Ocimum x africanum 100a in capsule 401. This capsule 401 can be administered to a person, allowing the tulsi extracts 101 within Ocimum x africanum 100a to bind to and inhibit the activity of SC2 Main Protease (6Y84) within the person. Capsule 401 may also contain fenugreek seeds 200 and neem 300 to enhance the binding and inhibition process. The contents of capsule 401 can be in powder form or be broken or shredded leaves and/or seeds respectively.

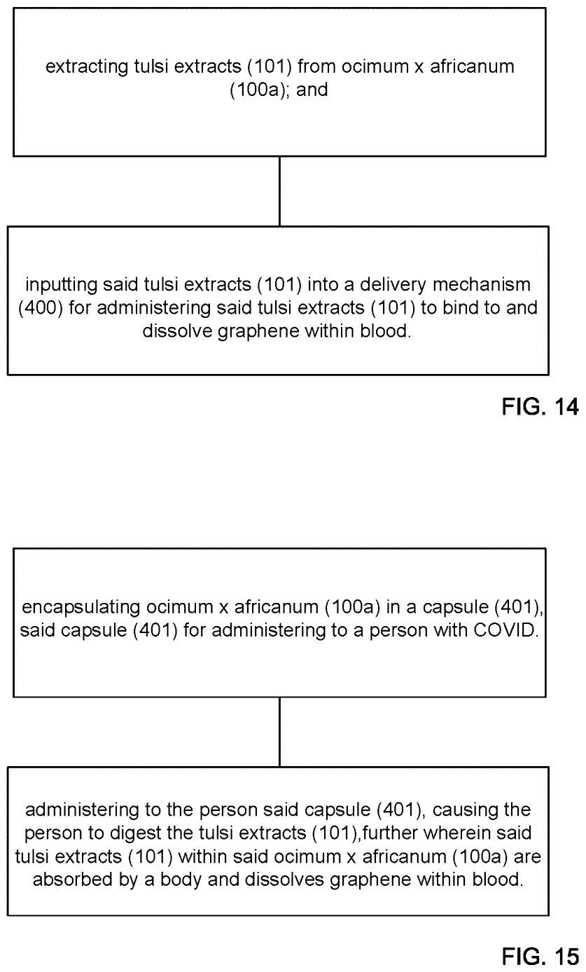

FIG. 14 illustrates a method for dissolving graphene in blood. This method involves extracting tulsi extracts from tulsi 100 such as Ocimum x africanum 100a and inputting these extracts into a delivery mechanism 400 for administering the tulsi extracts 101 to bind to and dissolve graphene within the blood. The extraction process includes boiling tulsi 100, and the delivery mechanism 400 can be the water used in boiling, forming tea 501. Administering the tulsi extracts 101 involves the person drinking the tea 501. Alternatively, the delivery mechanism 400 can be intravenous (IV) fluid 601.

FIG. 15 illustrates a second method for dissolving graphene in blood. Another method for dissolving graphene in blood involves encapsulating tulsi 100 such as Ocimum x africanum 100a in capsule 401. This capsule 401 is administered to a person, allowing the tulsi extracts 101 within tulsi 100 to dissolve graphene within the blood. Capsule 401 may also contain fenugreek seeds 200 and neem 300 to enhance the dissolving process. The tulsi 100 can be in powder form or in the form of broken or chopped leaves.

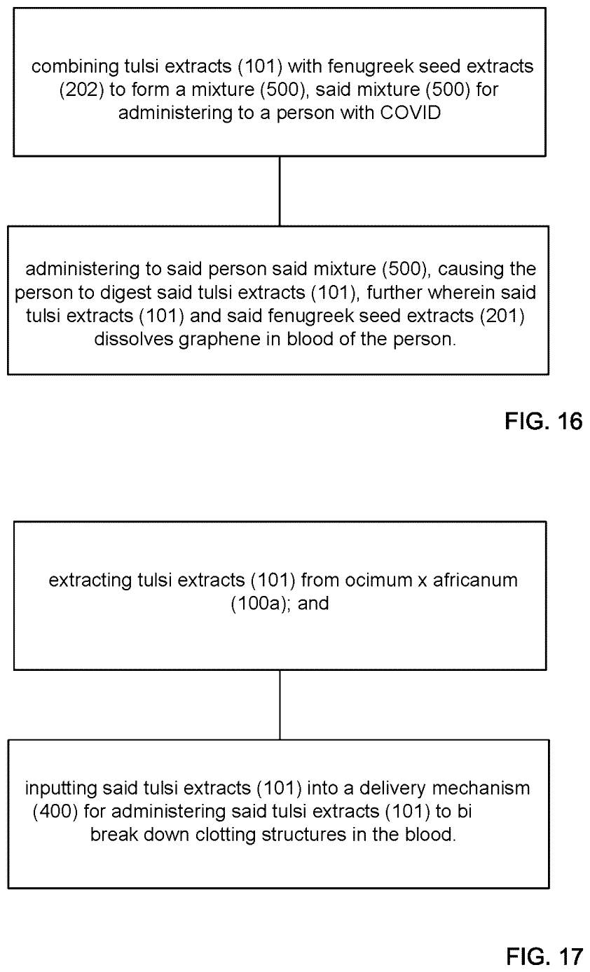

FIG. 16 illustrates a third method for dissolving graphene in blood. A further method for dissolving graphene in blood involves combining tulsi extracts 101 with fenugreek seed extracts 201 to form a mixture 500. This mixture 500 is administered to a person to dissolve graphene in the blood. The preferred ratio for this mixture is 2:1. Additionally, neem leaf extracts 301 can be added to the mixture 500 to enhance the dissolving process. The preferred ratio for this mixture is 4:2:1. The mixture can be administered in various forms, including ground leaves and seeds, dried and broken leaves, or suspended in intravenous (IV) fluid 601. For ease of administration, the mixture 500 can be encapsulated in capsule 401 or tea bag. The IV fluid 601 option provides a direct and efficient delivery mechanism 400, ensuring that the active compounds are rapidly absorbed into the bloodstream.

FIG. 17 illustrates a method for breaking down clotting structures in blood. This method involves extracting tulsi extracts 101 from tulsi 100 such as Ocimum x africanum 100a and inputting these extracts into a delivery mechanism 400 for administering the tulsi extracts 101 to bind to and break down clotting structures in the blood. The extraction process includes boiling the tulsi 100, and the delivery mechanism 400 can be the water used in boiling, forming a tea 501. Administering the tulsi extracts 101 involves the person drinking the tea 501. Alternatively, the delivery mechanism 400 can be intravenous (IV) fluid 601.

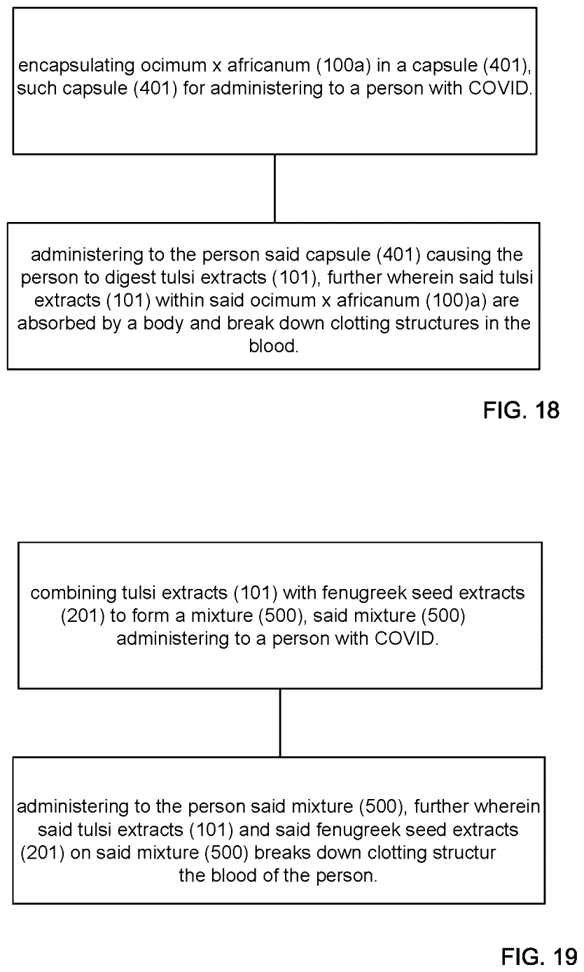

FIG. 18 illustrates a second method for breaking down clotting structures in blood. Another method for breaking down clotting structures in blood involves encapsulating tulsi 100 such as Ocimum x africanum 100a in capsule 401. This capsule 401 is administered to a person, allowing the tulsi extracts 101 within the tulsi 100 to break down clotting structures in the blood. Capsule 401 may also contain fenugreek seeds 200 and neem 300 to enhance the breaking down process. The tulsi can be in powder form or in the form of broken or chopped leaves.

FIG. 19 illustrates a third method for breaking down clotting structures in blood. A further method for breaking down clotting structures in blood involves combining tulsi extracts 101 with fenugreek seed extracts 201 to form a mixture 500. This mixture 500 is administered to a person to break down clotting structures in the blood. The preferred ratio for this mixture is 2:1. Additionally, neem leaf extracts 301 can be added to the mixture 500 to enhance the breaking down process. The preferred ratio for this mixture is 4:2:1. The mixture 500 can be administered in various forms, including ground leaves and seeds, dried and broken leaves, or suspended in intravenous (IV) fluid 601. For ease of administration, the mixture 500 can be encapsulated in capsule 401 or tea bag. The IV fluid option provides a direct and efficient delivery mechanism 400, ensuring that the active compounds are rapidly absorbed into the bloodstream.

These detailed descriptions provide a comprehensive understanding of the system and methods for reducing oxidative stress in the blood, inhibiting the activity of SARS-CoV-2 proteins, dissolving graphene in the blood, and breaking down clotting structures in the blood, ensuring that all aspects of the invention are fully disclosed. The provided alternatives and measurement tolerances enhance the flexibility and accessibility of the methods, allowing for a broader range of applications and user preferences. Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein”.

1. A method for binding to and inhibiting the activity of the SC2 RNA-dependent RNA polymerase (6M71) comprising
extracting tulsi extracts from Ocimum x africanum; and
inputting said tulsi extracts into a delivery mechanism for administering to a person said tulsi extracts to bind to and inhibit the activity of SC2 RNA-dependent RNA polymerase (6M71) within said person.

2. The method of claim 1 wherein extracting tulsi extracts comprises boiling said Ocimum x africanum.

3. The method of claim 2 wherein said delivery mechanism is water used in said boiling, said water and said tulsi extracts together at least part of a tea.

4. The method of claim 3 wherein administering to a person said tulsi extracts comprises said person drinking the tea.

5. The method of claim 1 wherein said delivery mechanism is intravenous (IV) fluid.

6. A method for binding to and inhibiting the activity of the SC2 RNA-dependent RNA polymerase (6M71) comprising
encapsulating Ocimum x africanum in a capsule, such capsule for administering to a person such that tulsi extracts within said Ocimum x africanum bond to and inhibit the activity of SC2 RNA-dependent RNA polymerase (6M71) within the person.

7. The method of claim 5 further comprising encapsulating fenugreek seeds in said capsule with said Ocimum x africanum.

8. The method of claim 6 further comprising encapsulating neem in said capsule with said Ocimum x africanum and said fenugreek seeds.

9. The method of claim 5 wherein said Ocimum x africanum is in a powder form.

10. The method of claim 5 wherein said Ocimum x africanum is from leaves.
